(19) United States
(12) Patent Application Publication
(10) Pub. No.: US 2008/0216870 A1
(43) Pub. Date: Sep. 11, 2008

(54) Dry Ice Blasting With Ozone-Containing Carrier Gas

(75) Inventor: Ronald J. MERRITELLO, Palos Hts., IL (US)

Correspondence Address: AIR LIQUIDE;Intellectual Property, 2700 POST OAK BOULEVARD, SUITE 1800, HOUSTON, TX, 77056, US

(73) Assignee: Air Liquid Industrial U.S. LP, Houston, TX (US)

(21) Appl. No.: 12/016,452

(22) Filed: Jan. 18, 2008

Related U.S. Application Data
(60) Provisional application No. 60/885,680, filed on Jan. 19, 2007.

(51) Int. Cl. B08B 7/02; B08B 9/02; A61L 2/22; A61L 2/02

(52) U.S. Cl. 134/8; 134/7; 422/293; 422/186.08; 422/186.07

(57) ABSTRACT

A surface of a target item is treated by directing a flow of solid carbon dioxide entrained in ozone towards it.

### CROSS-REFERENCE TO RELATED APPLICATIONS

This application claims the benefit under 35 U.S.C. § 119(e) to provisional application No. 60/885,680, filed Jan. 19, 2007, the entire contents of which are incorporated herein by reference.

### BACKGROUND

Dry ice is generally used for many applications, like cooling and chilling purposes in the food and beverage industries. One of the latest uses is the use of dry ice in blasting applications. Dry ice blasting is similar to sandblasting, high-pressure water blasting, or steam blasting. Dry ice blasting systems may project flakes (dry ice snow) or small rice size pellets (dry ice pellets) of dry ice at a temperature of about −78° C. out of a jet nozzle or applicator together with compressed air onto the surface of a target material. The low dry ice temperature causes contaminants on the target material surface to shrink and loose adhesion to the target material. The warmer sub surface of the target material causes the dry ice to sublime into carbon dioxide gas which has about 800 times greater volume than the solid dry ice. The carbon dioxide expands behind the contaminant speeding up contaminant removal from the surface. The contaminant then typically falls to the ground, or into some receptacle. Because the dry ice evaporates, only the contaminant is left for disposal.

Because conventional dry ice blasting provides cleaning based on using flakes or pellets of solid carbon dioxide alone, the potential applications, and/or the effectiveness for a given application, are limited. Therefore, the remains a need for improving and/or expanding the uses dry ice blasting.

### SUMMARY

A first embodiment of the invention is directed to a method of treating a surface of a target item that includes the following steps. A source of carbon dioxide is provided. A pressurized carrier gas comprising ozone is provided. A flow of solid carbon dioxide entrained in ozone is formed from the carbon dioxide source and carrier gas. The flow is directed to the target item surface.

A second embodiment of the invention is directed to a system for treating a surface of a target item that includes: a source of carbon dioxide; a source of a pressurized carrier gas comprising ozone; and an assembly fluidly communicating with the sources of carbon dioxide and carrier gas, the assembly being adapted and configured to produce a flow of solid carbon dioxide entrained in ozone from the sources of carbon dioxide and carrier gas.

Either or both of these embodiments may include one or more of the following aspects:
the carrier gas comprises ozonated air.
the carrier gas is formed by feeding an oxygen-containing gas to an ozone generator to thereby produce the carrier gas wherein the oxygen-containing gas is not air.
oxygen and nitrogen are blended to form synthetic air and feeding the synthetic air to an ozone generator to thereby produce ozonated synthetic air as the carrier gas.
the solid carbon dioxide is configured as a plurality of pellets.
the solid carbon dioxide is configured as a plurality of flakes.
the target item is food processing equipment.
the target item has a smoky odor.
the target item is a moldy building structure
the source of carbon dioxide comprises solid pellets of carbon dioxide.
the source of carbon dioxide comprises solid pellets of carbon dioxide and the assembly includes
   a mixing chamber having a pellet inlet, a carrier gas inlet, and a mixing chamber outlet;
   a first conduit having a first end in fluid communication with the carbon dioxide source and a second end;
   first valve adapted and configured to allow or prevent fluid communication between the first conduit second end and the pellet inlet
   a second conduit having a first end in fluid communication with the carrier gas source and a second end;
   a second valve adapted and configured to allow or prevent fluid communication between the second conduit second end and the carrier gas inlet;
   a controller adapted and configured to selectively open and close the first and second valves to provide a near-continuous supply of carbon dioxide pellets and carrier gas to the mixing chamber;
   a tube having a first end fluidly communicating with the mixing chamber outlet and a second end; and
   a nozzle in fluid communication with, and being disposed at, the second end of the tube.
the source of carbon dioxide comprises solid pellets of carbon dioxide and the assembly includes:
   a first tube having a first end in fluid communication with the source of carbon dioxide and a second end;
   a second tube having a first end in fluid communication with the source of carrier gas and a second end;
   a mixing chamber in fluid communication with the second end of the first tube; and
   a nozzle disposed within an interior of the mixing chamber and being in fluid communication with the second tube second end, wherein the second tube extends partially into said interior and terminates at the nozzle.
the source of carbon dioxide comprises liquid carbon dioxide.
the source of carbon dioxide comprises liquid carbon dioxide and the assembly includes
   a first conduit having a first end in fluid communication with the carbon dioxide source and second end;
   a second conduit having a first end in fluid communication with the carrier gas source and a second end;
   an outer tube having first and second ends;
   an inner tube extending along the outer tube and being concentrically disposed therein and having an inlet and an outlet;
   a nozzle disposed within the inner tube adjacent the inner tube outlet, wherein
      said second tube second end is in fluid communication with an annular gap formed between the tubes,
      said first tube second end is in fluid communication with the inlet, and
      said annular gap is open adjacent the outlet.
the source of carbon dioxide comprises liquid carbon dioxide and assembly includes:
   a first conduit having a first end in fluid communication with the carbon dioxide source and second end;
   a second conduit having a first end in fluid communication with the carrier gas source and a second end;
   an outer tube having an outlet;
   an inner tube extending partially along the outer tube and being concentrically disposed therein and having first and second ends, the inner tube first end being in fluid communication with the first conduit second end; and
   a diffuser disposed at the inner tube second end being adapted and configured to allow liquid carbon dioxide to flow from the inner tube second end through a plurality of apertures formed in the diffuser and into the outer tube.

### BRIEF DESCRIPTION OF THE DRAWINGS

For a further understanding of the nature and objects of the present invention, reference should be made to the following detailed description, taken in conjunction with the accompanying drawings, in which like elements are given the same or analogous reference numbers and wherein:

FIG. 1 is a schematic of a so-called “one-tube” embodiment of the invention utilizing a solid carbon dioxide feed;

FIG. 2 is a schematic of a so-called “two-tube” ventouri embodiment of the invention utilizing a solid carbon dioxide feed.

FIG. 3 is a schematic of a two component concentric nozzle embodiment of the invention utilizing a liquid carbon dioxide feed.

FIG. 4 is a schematic of diffuser embodiment of the invention utilizing a liquid carbon dioxide feed.

### DESCRIPTION OF PREFERRED EMBODIMENTS

The words and phrases used herein should be given their ordinary and customary meaning in the art by one skilled in the art unless otherwise further defined.

In the following, reference is made to embodiments of the invention. However, it should be understood that the invention is not limited to specific described embodiments. Instead, any combination of the following features and elements, whether related to different embodiments or not, is contemplated to implement and practice the invention. Furthermore, in various embodiments the invention provides numerous advantages over the prior art. However, although embodiments of the invention may achieve advantages over other possible solutions and/or over the prior art, whether or not a particular advantage is achieved by a given embodiment is not limiting of the invention. Thus, the following aspects, features, embodiments and advantages are merely illustrative and are not considered elements or limitations of the appended claims except where explicitly recited in a claim(s). Likewise, reference to “the invention” shall not be construed as a generalization of any inventive subject matter disclosed herein and shall not be considered to be an element or limitation of the appended claims except where explicitly recited in a claim(s).

As used herein, the phrase “target item” or “target material” refers to equipment, devices, structures, food products, pharmaceutical products, or other items that are in need of surface treatment, sanitation, preserving, deodorization, or otherwise protecting from or treated for pathogenic microorganisms. Equipment includes but is not limited to food processing equipment such as wafer plates, waffle irons, baker molds, ovens, conveyor belts, storage racks, trays, flights, tanks, screws and augers. Structures include but are not limited to building structures that are moldy or have a smoky odor.

Dry ice blasting technologies utilizing a compressed gas as a carrier gas are well known in the art, and it is believed that any system or apparatus suitable for such blasting is capable of use with the invention so long as an ozone-containing gas is used as the carrier gas. Generally, there are two types of dry ice blasting. The first type, pellet blasting, utilizes a dry ice pellet feed and produces a stream of dry ice pellets and ozone. The second type, snow blasting, utilizes a liquid carbon dioxide feed and produces a stream of dry ice flakes (snow) and ozone.

Pellet blasting equipment suitable for retrofitting for use in the invention may be obtained from the following companies: Ice Sonic located in Gauteng South Africa; Precision Iceblast Corporation located in Wallace, Mich., USA; Phoenix Unlimited LLC located in Corona, Calif., USA; CMW CO₂ Technologies located in Bandra (W), Mumbai, INDIA; Triventek Inc. located in Chicago, Ill., USA; IceTech A/S located in Bramming, Denmark; Asco Carbon Dioxide, Ltd. located in Romanshorn, Switzerland; BDI Canada CO2 Technologies located in Brampton, Ontario, Canada. These devices typically include a compressed air line connected to a mixing chamber. They may be retrofitted by replacing the compressed air line with a hose connected to an outlet of an ozone generator. Depending upon the concentration of ozone desired and the concentration of ozone achievable by the ozone generator, the ozone may be diluted with a different compressed gas, such as air, synthetic air, or nitrogen. This is easily accomplished by inserting a tee connection in the hose or other conventional fittings.

Snow blasting equipment suitable for retrofitting for use in the invention may be obtained from the following companies: Advanced Clean Production GmbH located in Esslingen, Germany; and Applied Surface Technologies located in New Providence, N.J., USA. These devices typically include a compressed air line or gaseous nitrogen line that feeds air or nitrogen to a periphery of the nozzle through which dry ice flakes are ejected thereby forming a shroud around the flakes being directed to the target. In each case, the inlet hose for compressed air or gaseous nitrogen is replaced with a hose connected to an outlet of an ozone generator. Depending upon the concentration of ozone desired and the concentration of ozone achievable by the ozone generator, the ozone may be diluted with a different compressed gas, such as air, synthetic air, or nitrogen. This is easily accomplished by inserting a tee connection in the hose or other conventional fittings.

A wide variety of ozone generators are commercially available having a wide range of ozone outputs and pressures, including those obtainable from Ozonia North America located in Elmwood Park, N.J., USA. Generally speaking, though, an ozone generator for use in the invention may be selected based upon the pressure requirement of the blasting equipment. If the maximum pressure of a given ozone generator is less than that required by the blasting equipment, a compressor may be inserted in between the outlet of the generator and the inlet of the snow-producing nozzle in order to achieve the desired pressure level.

Different types of ozone-containing carrier gases may be used, including but not limited to, ozonated air; “synthetic air” (a mixture of N₂ and O₂ with or without Ar); oxygen (concentration of about 93%) produced by a vapor swing adsorption (VSA) unit; and mixtures thereof. Ozonated air is preferred for simplicity's sake. If desired, the carrier gas may be pre-cooled by heat exchange with liquid nitrogen. Typical ozone concentrations produced by the ozone generator range from as low as about 3%, such as that produced with an air feed, to about 15%, such as that produced with an oxygen feed. One of ordinary skill in the art will recognize that oxygen-enriched air may also be used as the feed. In such case, the result ozone-containing gas produced by the generator will have an ozone concentration in between 3% and 15%.

The carrier gas need not have the same ozone concentration as that of the ozone-containing gas produced by the ozone generator. If desired, the ozone-containing gas at the outlet of the ozone generator may be diluted with air, synthetic air, or nitrogen.

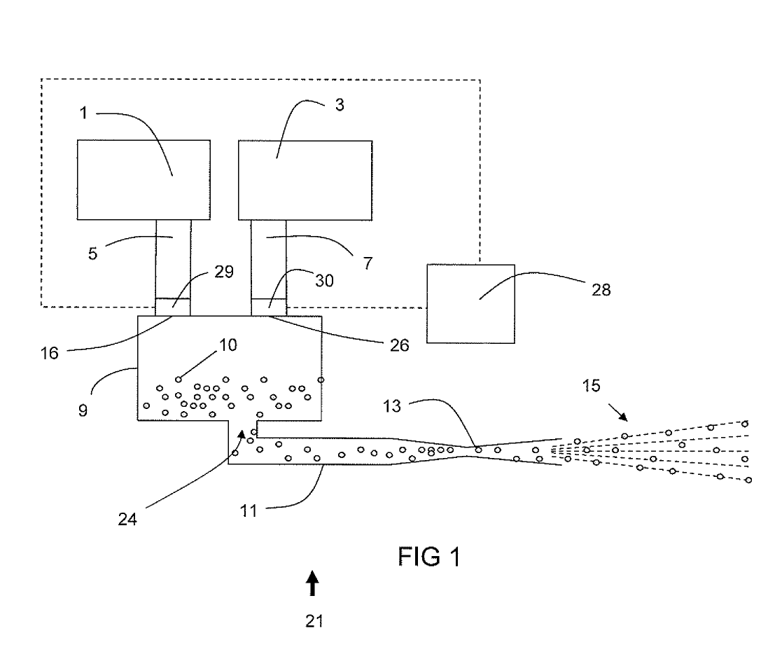

As best illustrated in FIG. 1, in a so-called “single tube” embodiment, an apparatus 21 comprises first conduit 5, second conduit 7, first valve 29, second valve 30, mixing chamber 9, tube 11, controller 28, and converging-diverging nozzle 13. Controller 28 opens first valve 29 and closes second valve 30 thereby allowing carbon dioxide pellets 10 to be either gravity fed from carbon dioxide pellet source 1 or fed with the aid of a compressed gas such as air to mixing chamber 9 via conduit 5 and pellet inlet 16. Controller 28 closes first valve 29 to seal off mixing chamber 9 from conduit 5 at pellet inlet 16. Controller 28 opens second valve 30 thereby allowing pressurized ozone-containing carrier gas from carrier gas source 3 to be supplied to chamber 9 via conduit 7 and carrier gas inlet 26. The pressure of the carrier gas forces the pellets 10 through mixing chamber outlet 24 and into tube 11 to converging-diverging nozzle 13. A flow 15 of carbon dioxide pellets 10 and ozone is ejected from the outlet of the nozzle 13. Controller 28 selectively opens and closes valves 29, 30 to recharge mixing chamber 9 with pellets and ozone and provide a near-continuous supply of flow 15.

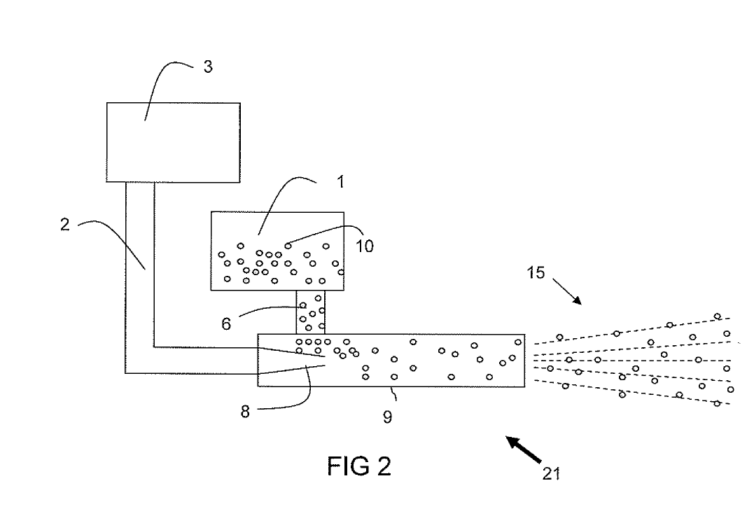

As best illustrated in FIG. 2, in a so-called “two tube” embodiment, an assembly 21 comprises first tube 6, second tube 2, mixing chamber 9, and nozzle 8. Carbon dioxide pellets 10 are fed from carbon dioxide pellet source 1 to mixing chamber 9 via tube 6. At the same time, ozone-containing carrier gas is supplied to nozzle 8 via tube 2. The flow of carrier gas accelerates through nozzle 8 and is expanded into an interior of chamber 9. A ventouri effect created by nozzle 8 entrains pellets 10 into flow 15 ejected at an outlet of chamber 9.

The dry ice pellets 10 typically have sizes in the range of 1/16 inch to 1 inch. They may be produced off-site and stored in a hopper in which case source 1 comprises an insulated hopper. Alternatively, they may be produced onsite at a pelletizer in which case source 1 comprises a pelletizer and an insulated hopper receiving the pellets 10 from the pelletizer. The pellets are then fed from the hopper to the pellet inlet of the mixing chamber 9.

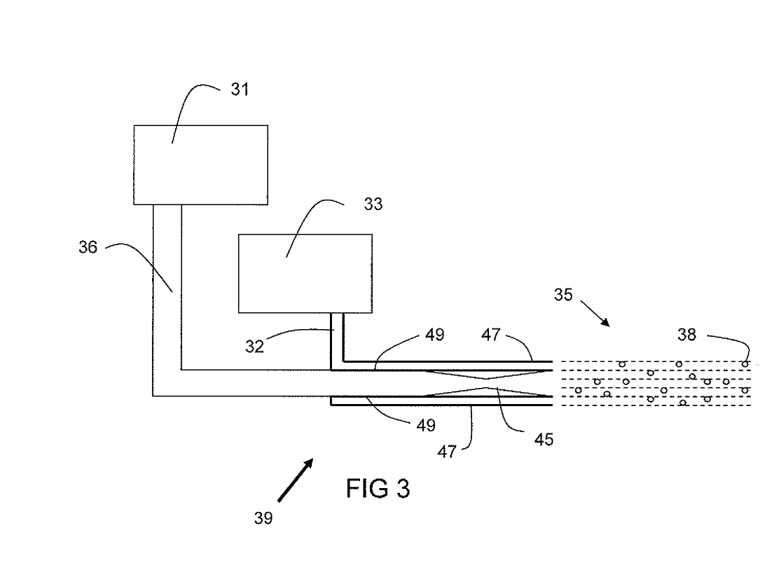

As best shown in FIG. 3, in a two component concentric nozzle embodiment, an assembly 39 comprises a first conduit 36, a second conduit 32, an outer tube 47, an inner tube 49, and a converging-diverging nozzle 45. A source of liquid carbon dioxide 31 supplies liquid carbon dioxide to inner tube 49 via conduit 36. At the same time, carrier gas comprising ozone is supplied to an annular gap formed between said inner and outer tubes 49, 47 from carrier gas source 33 via conduit 32. The carrier gas flows through the annular gap and is ejected adjacent an outlet of the nozzle 45. Liquid carbon dioxide expands at the outlet of the nozzle 45 and forms flakes of solid carbon dioxide (snow) 38 where it is entrained within a high velocity jet of carrier gas to form flow 35. Typically, the pressure, flow rate, and annular gap are sized to provide a carrier gas flow rate at velocities in a range from just below sonic to above sonic.

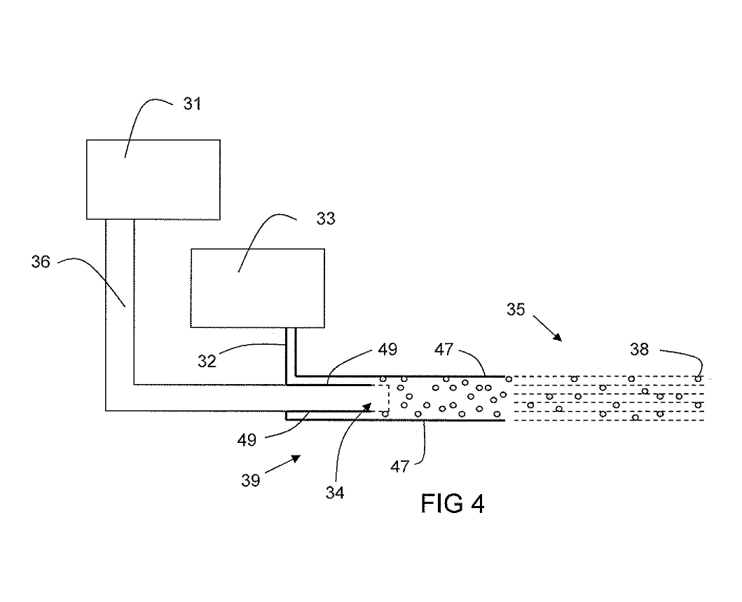

As best illustrated in FIG. 4, in a diffuser embodiment, assembly 39 comprises a first conduit 36, a second conduit 32, an outer tube 47, an inner tube 49, and a diffuser 34. A source of liquid carbon dioxide 31 supplies liquid carbon dioxide to inner tube 49 via conduit 36. At the same time, carrier gas comprising ozone is supplied to an interior of outer tube 47 from carrier gas source 33 via conduit 32. The inner tube 49 is arranged concentrically within outer tube 47 and extends partially therealong. Liquid carbon dioxide flows through inner tube 49 and through apertures formed in diffuser 34 where it expands and forms solid flakes of solid carbon dioxide 38 which are propelled forward by the carrier gas flowing in outer tubes 47 to thereby form flow 35.

Preferred processes and apparatus for practicing the present invention have been described. It will be understood and readily apparent to the skilled artisan that many changes and modifications may be made to the above-described embodiments without departing from the spirit and the scope of the present invention. The foregoing is illustrative only and that other embodiments of the integrated processes and apparatus may be employed without departing from the true scope of the invention defined in the following claims.

What is claimed is:

1. A method of treating a surface of a target item, comprising the steps of:
providing a source of carbon dioxide;
providing a pressurized carrier gas comprising ozone;
producing a flow of solid carbon dioxide entrained in ozone from said carbon dioxide source and carrier gas;
directing the flow to the target item surface.

2. The method of claim 1, wherein the carrier gas comprises ozonated air.

3. The method of claim 1, wherein the carrier gas is formed by feeding an oxygen-containing gas to an ozone generator to thereby produce the carrier gas wherein the oxygen-containing gas is not air.

4. The method of claim 1, further comprising the steps of blending oxygen and nitrogen to form synthetic air and feeding the synthetic air to an ozone generator to thereby produce ozonated synthetic air as the carrier gas.

5. The method of claim 1, wherein the solid carbon dioxide is configured as a plurality of pellets.

6. The method of claim 1, wherein the solid carbon dioxide is configured as a plurality of flakes.

7. The method of claim 1, wherein the target item is food processing equipment.

8. The method of claim 1, wherein the target item has a smoky odor.

9. The method of claim 1, wherein the target item is a moldy building structure.

10. A system for treating a surface of a target item, comprising:
a source of carbon dioxide;
a source of a pressurized carrier gas comprising ozone; and
an assembly fluidly communicating with said sources of carbon dioxide and carrier gas, said assembly being adapted and configured to produce a flow of solid carbon dioxide entrained in ozone from said sources of carbon dioxide and carrier gas.

11. The system of claim 10, wherein said source of carbon dioxide comprises solid pellets of carbon dioxide.

12. The system of claim 11, wherein said assembly comprises:
a mixing chamber having a pellet inlet, a carrier gas inlet, and a mixing chamber outlet;
a first conduit having a first end in fluid communication with said carbon dioxide source and a second end;
a first valve adapted and configured to allow or prevent fluid communication between said first conduit second end and said pellet inlet
a second conduit having a first end in fluid communication with said carrier gas source and a second end;
a second valve adapted and configured to allow or prevent fluid communication between said second conduit second end and said carrier gas inlet;
a controller adapted and configured to selectively open and close said first and second valves to provide a near-continuous supply of carbon dioxide pellets and carrier gas to said mixing chamber;
a tube having a first end fluidly communicating with said mixing chamber outlet and a second end; and
a nozzle in fluid communication with, and being disposed at, said second end of said tube.

13. The system of claim 11, wherein said assembly comprises:
a first tube having a first end in fluid communication with said source of carbon dioxide and a second end;
a second tube having a first end in fluid communication with said source of carrier gas and a second end;
a mixing chamber in fluid communication with said second end of said first tube; and
a nozzle disposed within an interior of said mixing chamber and being in fluid communication with said second tube second end, wherein said second tube extends partially into said interior and terminates at said nozzle.

14. The system of claim 10, wherein said source of carbon dioxide comprises liquid carbon dioxide.

15. The system of claim 14, wherein said assembly comprises:
a first conduit having a first end in fluid communication with said carbon dioxide source and second end;
a second conduit having a first end in fluid communication with said carrier gas source and a second end;
an outer tube having first and second ends;
an inner tube extending along said outer tube and being concentrically disposed therein and having an inlet and an outlet;
a nozzle disposed within said inner tube adjacent said inner tube outlet, wherein:
said second tube second end is in fluid communication with an annular gap formed between said tubes,
said first tube second end is in fluid communication with said inlet, and
said annular gap is open adjacent said outlet.

16. The system of claim 14, wherein said assembly comprises:
a first conduit having a first end in fluid communication with said carbon dioxide source and second end;
a second conduit having a first end in fluid communication with said carrier gas source and a second end;
an outer tube having an outlet;
an inner tube extending partially along said outer tube and being concentrically disposed therein and having first and second ends, the inner tube first end being in fluid communication with said first conduit second end; and
a diffuser disposed at said inner tube second end being adapted and configured to allow liquid carbon dioxide to flow from said inner tube second end through a plurality of apertures formed in said diffuser and into said outer tube.

17. The system of claim 10, wherein the carrier gas comprises ozonated air.

18. The system of claim 10, wherein the carrier gas is formed by feeding an oxygen-containing gas to an ozone generator to thereby produce the carrier gas wherein the oxygen-containing gas is not air.

19. The system of claim 10, wherein the carrier gas is ozonated synthetic air formed by blending oxygen and nitrogen to form synthetic air and feeding the synthetic air to an ozone generator.
